(19) United States
(12) Patent Application Publication
(10) Pub. No.: US 2016/0178594 A1
(43) Pub. Date: Jun. 23, 2016

(54) Analyzer system and method for sensing a chemical characteristic of a fluid sample

(71) Applicant: THERMO ORION INC., Chelmsford, MA (US)

(72) Inventors: George Jarvis, Arlington, MA (US); Victor Chung, Melrose, MA (US); Dave Balise, Townsend, MA (US); Edward Somes, Boxford, MA (US)

(21) Appl. No.: 14/757,760

(22) Filed: Dec. 23, 2015

Related U.S. Application Data
(60) Provisional application No. 62/095,870, filed on Dec. 23, 2014.

(51) Int. Cl. G01N 33/18; B01L 3/00; G01N 35/00; G01N 27/416

(52) U.S. Cl. CPC G01N 33/1886; G01N 27/4168; B01L 3/502; G01N 35/00871; B01L 2300/0867; B01L 2300/0645; B01L 2400/0622

(57) ABSTRACT

An analyzer system for sensing a chemical characteristic of a fluid sample according to one embodiment includes a sample inlet configured to receive the sample via the sample inlet, a reagent inlet configured to receive a reagent via the reagent inlet, a reaction zone in fluid communication with the sample inlet and the reagent inlet and being configured to receive the sample from the sample inlet and the reagent from the reagent inlet for mixing the sample with the reagent; and a sensor zone in fluid communication with the reaction zone, wherein the sensor zone comprises a sensor configured to sense the chemical characteristic of the mixed sample and reagent, and further wherein the sensor zone is configured to allow automatic drainage of the mixed sample and reagent away from the sensor after the sensor has sensed the chemical characteristic of the mixed sample and reagent.

### CROSS-REFERENCE TO RELATED APPLICATION

The present application claims the filing benefit of U.S. Provisional Application No. 62/095,870, filed Dec. 23, 2014, the disclosure of which is incorporated herein by reference in its entirety.

### FIELD OF THE INVENTION

The present invention generally relates to apparatuses and methods for sensing a chemical characteristic of a sample and, more particularly, to an analyzer system and method for sensing a chemical characteristic of a fluid in a fluid flow.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Processes in many industries include a treatment step for waste water generated during the process. For instance, cooling circuits in industrial plants often employ water prone to biofouling, and in other industrial settings, such as in large-scale shipping operations, the amount of organic material allowed to exist in the waste water, or ballast water, is typically limited by various applicable regulations. As a result, various water treatment protocols are known. Typical water treatment protocols involve the addition of chlorinated compounds, such as sodium hypochlorite and chlorine dioxide, to the water to disinfect any biological material present in the water. Although such a chlorine treatment is effective at mitigating the effects of biological materials, overuse or underuse of the chlorinated compound can lead to additional problems. For instance, costs of treatment are greatly increased when too much chlorinated compound is used. Additionally, the outflow of oxidant compounds from industrial processes is often regulated by governing bodies that set an upper limit on the amount of oxidants allowed in the outflow. On the other hand, if too little chlorinated compound is used, the treatment may be ineffective, leading to fouling of the process apparatus or non-compliance with the applicable regulations regarding outflow of biological materials.

As a result, many industries rely on the rapid and accurate measurement of the amount of residual oxidizing material remaining in a sample of water. In fresh water, measurement of the amount of chlorine in the sample is referred to as the Total Residual Chlorine concentration (hereinafter “TRC”), and in sea water, the same measurement is referred to as Total Residual Oxidant concentration (hereinafter “TRO”), owing to the presence of iodide and bromide ions in sea water. Applications as diverse as shipping vessels, water treatment plants, manufacturing centers, thermoelectric and nuclear power stations, oil extraction apparatuses, chemical plants, food production facilities, water pipelines, or any other application in which water is used for manipulating the local environment, all rely on rapid and accurate measurement of residual oxidizing material remaining in the water.

For example, the shipping industry is subject to many regulations, e.g. from the U.S. EPA, regarding the purity of the water expelled from ballast water tanks, regarding both un-neutralized organic materials and excess chlorinated compounds. In general, when a shipping vessel discharges its cargo at one port, it loads one or more ballast tanks with water adjacent to its hull to help stabilize the vessel. The water that is taken on remains in the ballast tanks until the ship arrives at the next port to take on cargo. As the cargo is loaded, the ballast tanks are emptied through ballast pipes or ducts, either partially or fully, because the ballast water is no longer necessary due to the added weight of the cargo. Because the ship will travel great distances between the two ports, current regulations require biocidal treatment of the water held in the ballast tanks, prior to the ballast water being discharged, to help prevent the proliferation of non-native species of organisms. Practical matters require a similar treatment protocol to remove biological material capable of leading to biofouling of the tanks. The treated water in the ballast tanks should be monitored to control the amount of chlorine added and to ensure that enough chlorine is added to treat the ballast water effectively.

Analogously, the applications listed above also require monitoring of the oxidant materials in the outflow of those applications. Therefore, there is a need for an analyzer system for sensing a chemical characteristic of a fluid sample which is both accurate in its measurement capability and readily adaptable for use in various environments and sample measurement applications.

### SUMMARY OF THE INVENTION

Certain exemplary aspects of the present invention are set forth below. It should be understood that these aspects are presented merely to provide the reader with a brief summary of certain forms the present invention might take and that these aspects are not intended to limit the scope of the present invention. Indeed, the present invention may encompass a variety of aspects that may not be explicitly set forth below.

In accordance with the principles of the present invention, and in the exemplary environment of a shipping vessel dumping ballast water into the proximate environment, an analyzer system according to one embodiment of the present invention may be installed after construction of the shipping vessel in many instances and may not require the input of any additional energy to sense a chemical characteristic of the flowing ballast water while it is being discharged from the shipping vessel. In other words, the analyzer system according to one embodiment of the present invention may be energetically self-contained. The analyzer system may be placed as close as possible to the ballast water outlet to ensure the highest quality measurement of the concentration of oxidant species at the location of its highest likelihood of environmental impact. Furthermore, the analyzer system according to one embodiment may be configured in such a manner that the sensor is protected from damage caused by standing sample water, such as ballast water, or by an analytical reagent used by the analyzer system, when the analyzer system is not in use.

According to one aspect of the present invention, an analyzer system is provided for sensing a chemical characteristic of a fluid sample. The analyzer system includes a sample inlet configured to receive a sample via the sample inlet; a reagent inlet configured to receive a reagent via the reagent inlet; a reaction zone in fluid communication with the sample inlet and the reagent inlet and being configured to receive the sample from the sample inlet and the reagent from the reagent inlet for mixing the sample with the reagent; and a sensor zone in fluid communication with the reaction zone. The sensor zone includes a sensor configured to sense the chemical characteristic of the mixed sample and reagent, and the sensor zone is configured to allow automatic drainage of the mixed sample and reagent away from the sensor after the sensor has sensed the chemical characteristic.

In another aspect of the present invention, the analyzer system includes a sample inlet configured to receive a sample via the sample inlet; a reagent inlet configured to receive a reagent via the reagent inlet; a sample reservoir in fluid communication with the sample inlet for receiving the sample from the sample inlet; an actuator having a first position and a second position, the actuator being configured to move from the first position to the second position in response to the sample being received in the sample reservoir; a reagent reservoir in fluid communication with the reagent inlet for receiving the reagent from the reagent inlet; a valve operatively coupled to the actuator being configured to release reagent from the reagent reservoir; a reaction zone in fluid communication with the sample reservoir and the reagent reservoir and being configured to receive the sample from the sample reservoir and the reagent from the reagent reservoir for mixing the sample with the reagent; and a sensor zone in fluid communication with the reaction zone. The sensor zone includes a sensor configured to sense the chemical characteristic of the mixed sample and reagent. Additionally, the reagent regulator is configured to release the reagent from the reagent reservoir in response to movement of the actuator from the first position to the second position.

In another aspect of the present invention, the analyzer system includes a sample inlet configured to receive a sample via the sample inlet; a reagent inlet configured to receive a reagent via the reagent inlet; an actuator having a first position and a second position, the actuator being configured to move from the first position to the second position in response to fluid flow; a reagent reservoir in fluid communication with the reagent inlet for receiving the reagent from the reagent inlet; a valve operatively coupled to the actuator being configured to release reagent from the reagent reservoir; a reagent outlet in fluid communication with the reagent reservoir; a reaction zone in fluid communication with the sample inlet and the reagent outlet and being configured to receive the sample from the sample inlet and the reagent from the reagent outlet for mixing the sample with the reagent; and a sensor zone in fluid communication with the reaction zone. The sensor zone includes a sensor configured to sense the chemical characteristic of the mixed sample and reagent. Additionally, the valve is configured to release the reagent from the reagent reservoir via the reagent outlet in response to movement of the actuator from the first position to the second position.

In another aspect of the present invention, a method is provided for monitoring the total residual oxidants present in a sample. The method includes providing the sample to the analyzer system of the present invention via the sample inlet; providing the reagent via the reagent inlet; mixing the sample and the reagent in the reaction zone; providing the mixed sample and reagent to the sensor zone; and sensing the total residual oxidants present in the sample.

According to yet another aspect, the present invention provides a method for sensing a chemical characteristic of a flowing fluid sample. The method includes, for example, providing the sample to an analyzer system; providing a reagent to the analyzer system; mixing the reagent and the sample; providing the mixed reagent and sample to a sensor zone; sensing a chemical characteristic of the sample; and removing the sample and reagent from the analyzer system. The power generated by the flowing fluid sample powers the steps of the method. Thus, no additional power is needed to sense the chemical characteristic.

The above and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention

FIG. 1 is a diagrammatic view of an exemplary analyzer system of the present invention shown installed in a ballast discharge duct of a shipping vessel.

FIG. 2 is a diagrammatic cross-sectional view of an analyzer system for sensing a chemical characteristic of a fluid sample according to one aspect of the present invention.

FIG. 2A is an enlarged view of the encircled area 2A shown in FIG. 2.

FIG. 2B is an end-on view of the analyzer system shown in FIG. 2.

FIG. 2C is a diagrammatic cross-sectional view taken along line 2C-2C of FIG. 2B.

FIG. 2D is a diagrammatic cross-sectional view of an analyzer system for sensing a chemical characteristic of a fluid sample according to an alternative aspect of the present invention

FIG. 3 is a diagrammatic cross-sectional view of an analyzer system for sensing a chemical characteristic of a sample according to a second aspect of the present invention.

FIG. 3A is an enlarged view of the encircled area 3A shown in FIG. 3.

FIG. 4 is a diagrammatic cross-sectional view of an analyzer system for sensing a chemical characteristic of a sample according to third aspect of the present invention.

FIG. 4A is an enlarged view of the encircled area 4A shown in FIG. 4.

FIG. 5 is a diagrammatic cross-sectional view of an analyzer system for sensing a chemical characteristic of a sample according to a fourth aspect of the present invention.

FIG. 5A is a diagrammatic view of the analyzer system of FIG. 5 mounted to the outside of a discharge duct.

FIG. 5B is a diagrammatic cross-sectional view taken along line 5B-5B of FIG. 5A.

FIG. 5C is a diagrammatic cross-sectional view of an analyzer system for sensing a chemical characteristic of a sample according to an alternative aspect of the present invention.

FIG. 5D is a diagrammatic view of the analyzer system of FIG. 5C mounted to the outside of a discharge duct.

FIG. 5E is a diagrammatic cross-sectional view taken along line 5E-5E of FIG. 5D.

FIG. 6 is a diagrammatic cross-sectional view of an analyzer system for sensing a chemical characteristic of a sample according to a fifth aspect of the present invention.

FIG. 6A is a diagrammatic cross-sectional view of an analyzer system for sensing a chemical characteristic of a sample according to an alternative aspect of the present invention.

FIG. 7 is a diagrammatic cross-sectional view of an analyzer system for sensing a chemical characteristic of a sample according to a sixth aspect of the present invention.

FIG. 7A is an enlarged view of the encircled area 7A shown in FIG. 7.

FIG. 8 is a diagrammatic cross-sectional view of an analyzer system for sensing a chemical characteristic of a sample according to a seventh aspect of the present invention.

FIG. 8A is an alternative embodiment of the analyzer system shown in FIG. 8.

FIG. 9 is a diagrammatic cross-sectional view of an analyzer system for sensing a chemical characteristic of a sample according to an eighth aspect of the present invention.

FIG. 9A shows the delivery of a cleaning solution to a sensor of the analyzer system shown in FIG. 9.

FIG. 10 is a diagrammatic cross-sectional view of an analyzer system for sensing a chemical characteristic of a sample according to a ninth aspect of the present invention.

FIG. 10A is a diagrammatic cross-sectional view of an analyzer system for sensing a chemical characteristic of a sample according to an alternative aspect of the present invention.

### DETAILED DESCRIPTION OF THE INVENTION

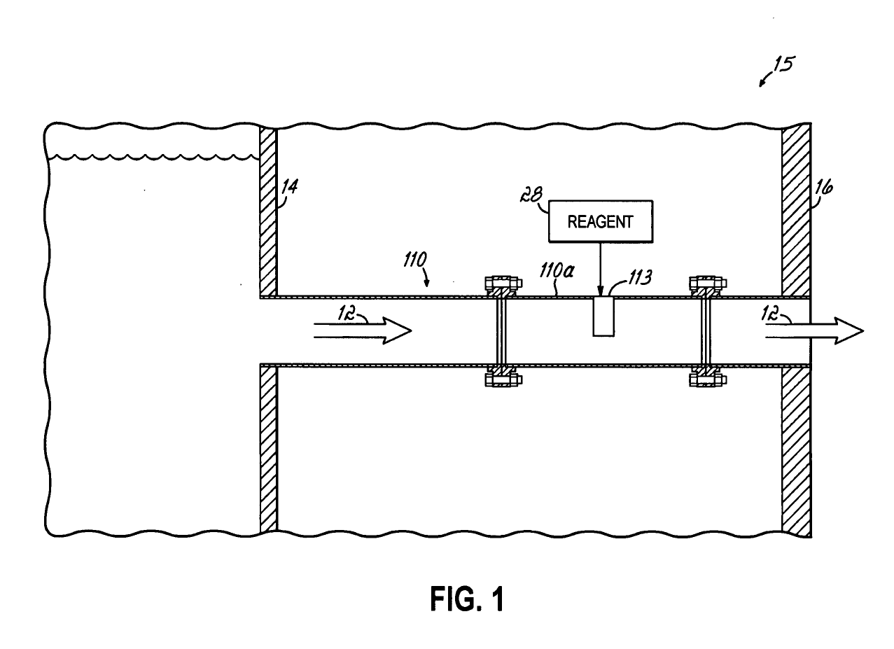

Referring now to the figures in which like numerals represent like parts, and to FIG. 1 in particular, an analyzer system 113 according to one embodiment of the present invention is shown installed in a ballast water discharge duct 110 located between a ballast tank 14 and a hull 16 of a shipping vessel 15. Analyzer system 113 may be positioned as close to hull 16 as possible to ensure the highest quality measurement of the concentration of oxidants in the ballast water at the location of its highest likelihood of impact to the surrounding environment. Reagent 28 is shown schematically and may exist in any convenient location within the confines of the present invention.

According to one embodiment as shown in FIG. 1, the analyzer system 113 is mounted in a duct section 110a which is installed in-line as a modular component of the ballast discharge duct 110. Of course, those of ordinary skill in the art will appreciate that other mountings and/or locations of the analyzer system 113 are possible, as well, without departing from the spirit and scope of the present invention.

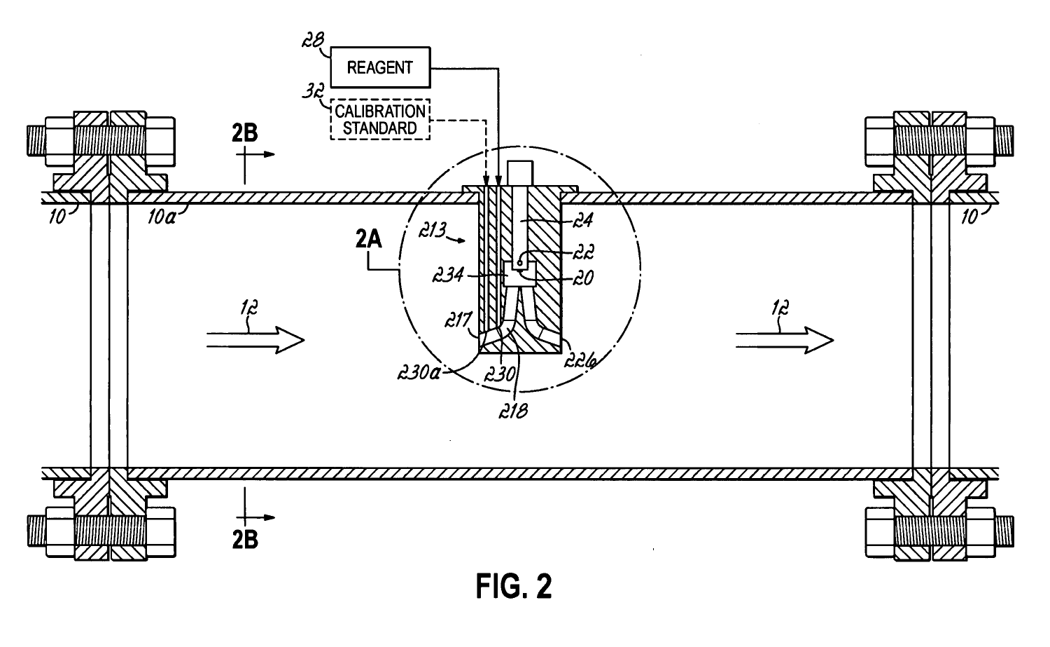

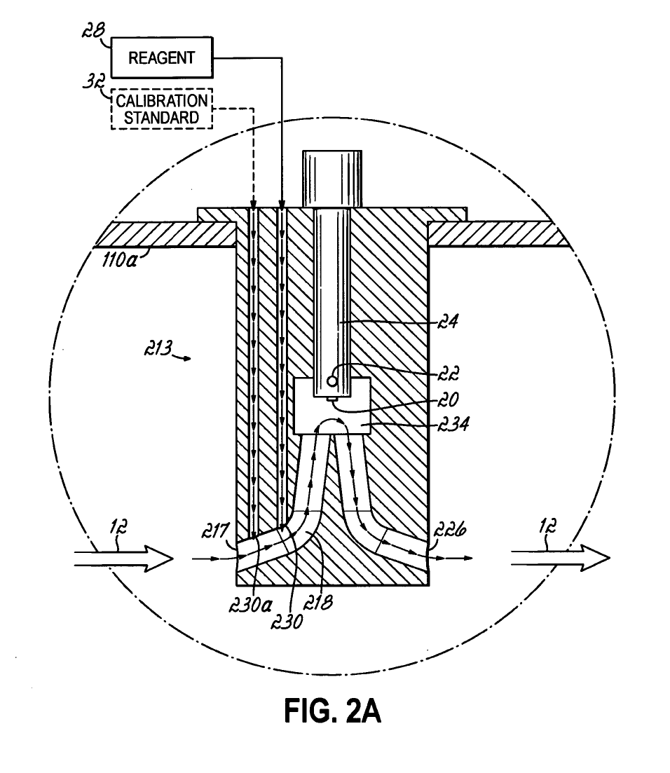

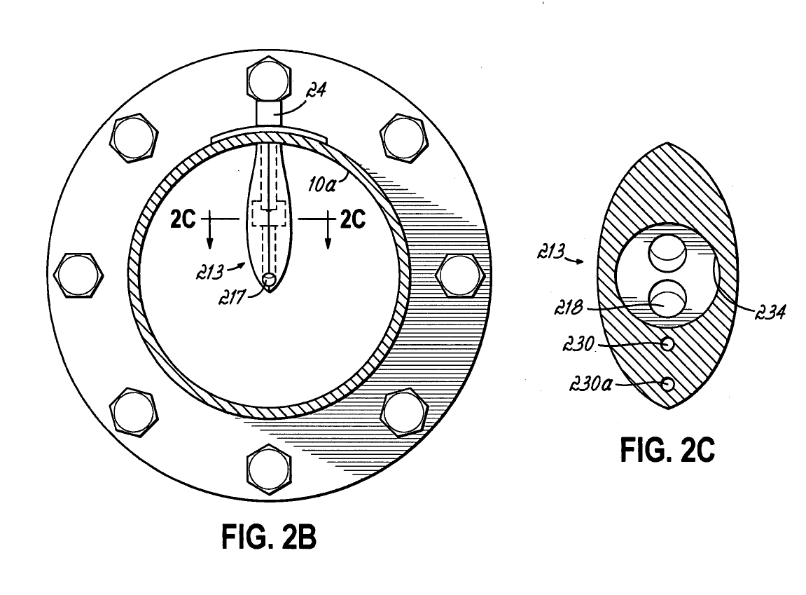

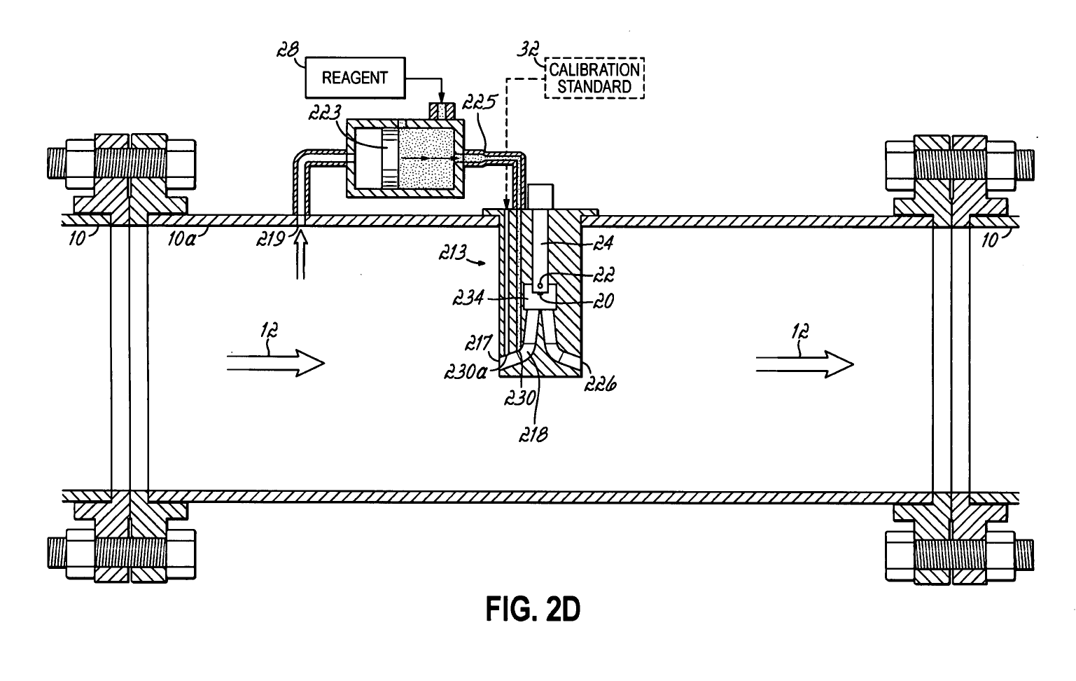

FIGS. 2 and 2A illustrate analyzer system 213 in greater detail, according to one embodiment of the present invention. In FIGS. 1-10A, process water flows from left to right, as designated by arrows 12. As the flow of the process water approaches sample inlet 217, a small volume of process water, i.e. the sample, enters sample inlet 217. Concurrently, the flow of the sample causes an actuator (not shown) to move, thereby driving the addition of reagent 28 through reagent inlet 230. The actuator may be pneumatic, in which case a diaphragm is depressed while a linkage opens a flow valve, or the actuator may be a mechanical linkage through the wall of the pipe. Alternatively, as shown in FIG. 2D, a portion of the process water may be diverted into inlet 219, the force of which pushes piston 223, which in turn forces reagent 28 into reagent inlet 230. In this alternative, flow restrictor 225 may be used to control the flow of reagent 28. The sample and reagent mix in reaction zone 218, and the flow of the mixed sample and reagent carry it upwardly in a vertical direction to sensor zone 234 where a chemical characteristic of the process water sample is sensed, as will be discussed in greater detail below. Sensor zone 234 includes at least one sensing electrode 20 and at least one reference electrode 22, which together comprise oxidants probe 24. The flow of process water in the discharge duct 10 pushes the mixed sample and reagent to outlet 226, which is positioned vertically downwardly from the sensor zone 234, where the mixture is expelled into the discharge duct 10. When the flow of process water in the discharge duct 10 ceases, the force of gravity causes the liquid species present in the sensor zone to drain into the discharge duct 10 via outlet 226. Optionally, a second probe (not shown) can be incorporated into sensor zone 234. The second probe may be, for example, a temperature probe or a pH probe.

Optionally, calibration standard inlet 230a may provide a defined amount of calibration standard 32. Calibration standard inlet 230a may operate with the same principals as reagent inlet 230. However, calibration standard inlet 230a may be coupled to a valve with a mechanical time delay such that opening of the valve is delayed by a fixed amount of time to allow detection of the added calibration standard. The calibration standard 32 may be added either in lieu of or in combination with process water.

FIGS. 2B and 2C show analyzer system 213 mounted in duct section 10a of the discharge duct 10. The outer housing of analyzer system 213 is shown in a contoured fin shape, which minimizes drag as the process water flows through the discharge duct 10. However, the analyzer system 213 may exist in housings of alternative shapes, and the present invention is not intended to be limited to only the contoured fin shape shown.

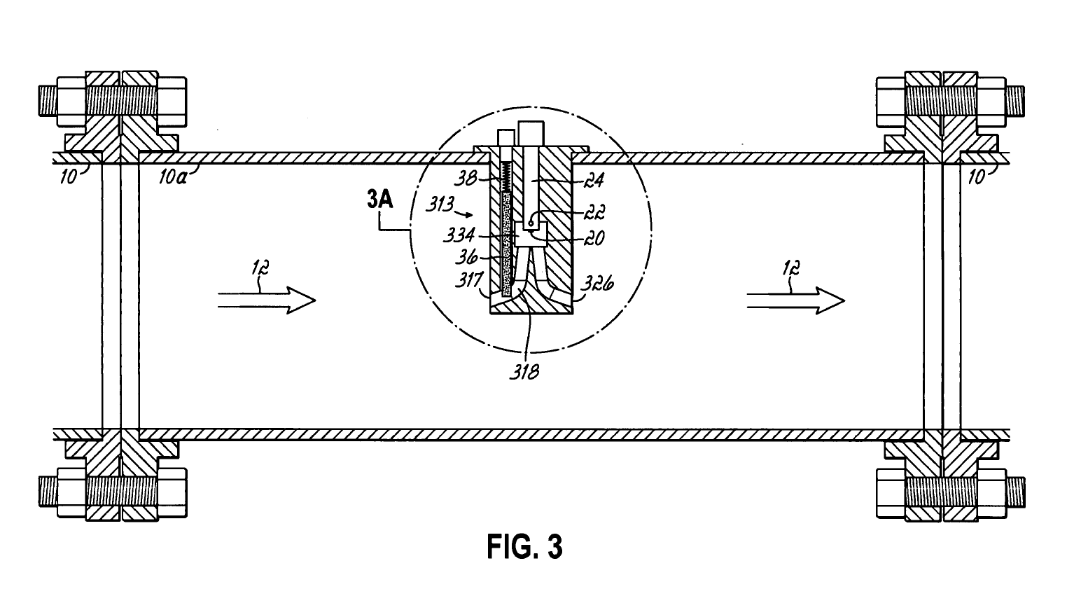

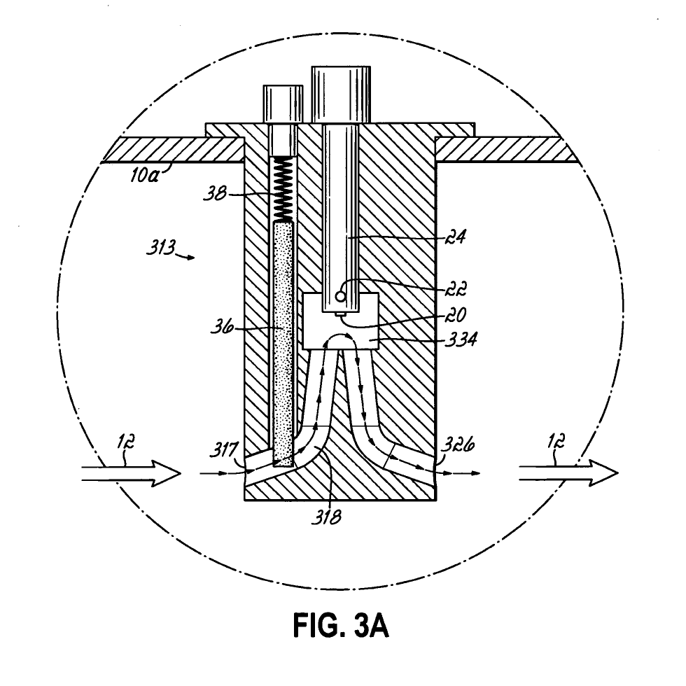

FIGS. 3 and 3A illustrate an analyzer system 313 according to a second embodiment of the present invention, shown mounted in the duct section 10a of the discharge duct 10 in which the reagent is a solid reagent. As the flow of the process water approaches sample inlet 317, a small volume of process water, i.e. the sample, enters sample inlet 317. Solid reagent 36 is held in the path of the sample flow by spring mechanism 38, which can be configured around or through the solid reagent 36. The solid reagent 36 can also be held in place by other mechanisms, such as gravity or a basket enclosure where the basket contains one or more solid pellets. As the sample flow interacts with the solid reagent 36, the sample and reagent mix in reaction zone 318. The mixed sample and reagent then flow into sensor zone 334 where a chemical characteristic of the process water sample is sensed. Sensor zone 334 includes at least one sensing electrode 20 and at least one reference electrode 22, which together comprise oxidants probe 24. The flow of process water in the discharge duct 10 pushes the mixed sample and reagent to outlet 326, which is positioned vertically downwardly from the sensor zone 334, where the mixture is expelled into the discharge duct 10. When the flow of process water in the discharge duct 10 ceases, the force of gravity causes the liquid species present in the sensor zone to drain into the discharge duct 10 via outlet 326. Optionally, a second probe (not shown) can be incorporated into sensor zone 334. The second probe may be, for example, a temperature probe or a pH probe.

Solid reagent 36 allows the system to require little maintenance, with the reagent needing to be replaced less frequently than approximately every six months, for example, although the present invention is not intended to be limited to such a time period.

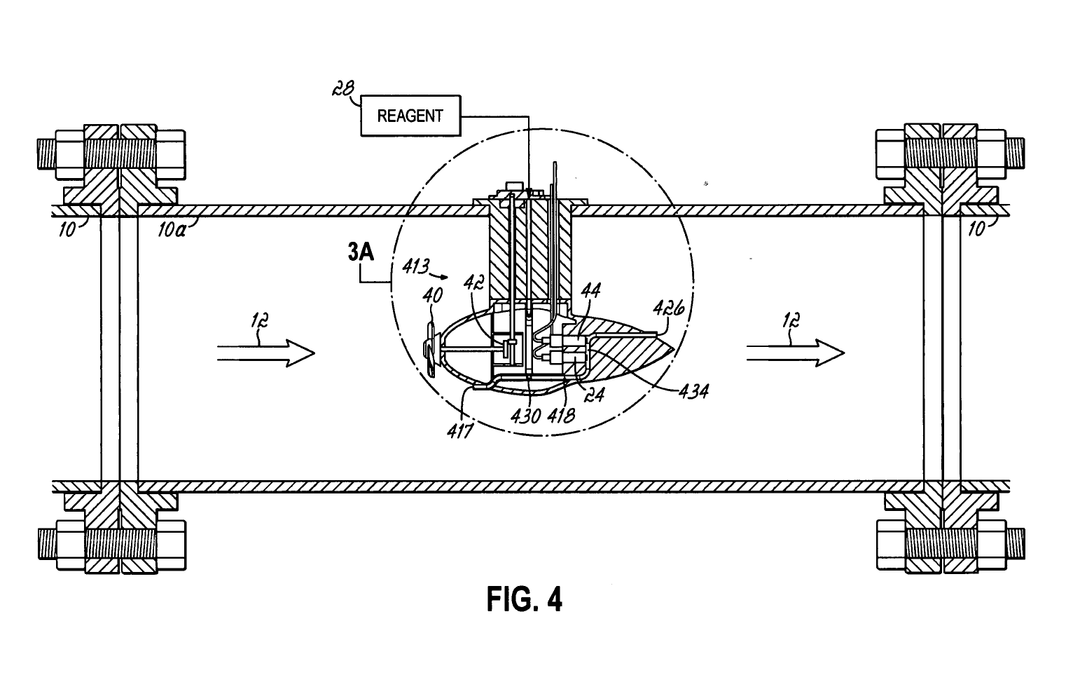

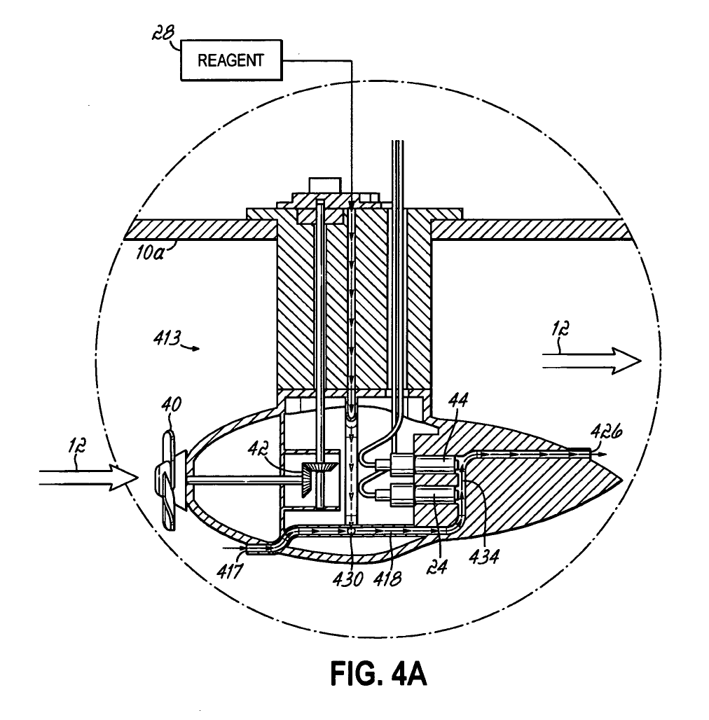

FIGS. 4 and 4A illustrate an analyzer system 413, according to a third embodiment of the present invention, shown mounted in the duct section 10a. As the flow of the process water approaches sample inlet 417, a small volume of process water, i.e. the sample, enters sample inlet 417. Concurrently, the flow of the process water causes propeller 40 to rotate. The rotation of propeller 40 drives transmission 42, which in turn activates the addition of reagent 28. Transmission 42 may turn a valve (not shown) or add pressure to a bellows (not shown), which urges reagent into reagent inlet 430. Alternatively, a portion of the process water may be diverted into an inlet to drive a piston to force reagent 28 into the system, analogous to the system shown in FIG. 2D. The sample and reagent mix in reaction zone 418, and the flow of the mixed sample and reagent carry it to sensor zone 434. Sensor zone 434 includes at least one oxidants probe 24. The flow of the sample and reagent subsequently carries the mixture to outlet 426, where the mixture is expelled into the discharge duct 10, via dynamic pressure. Optionally, second probe 44 can be incorporated into sensor zone 434. The second probe may be, for example, a temperature probe or a pH probe.

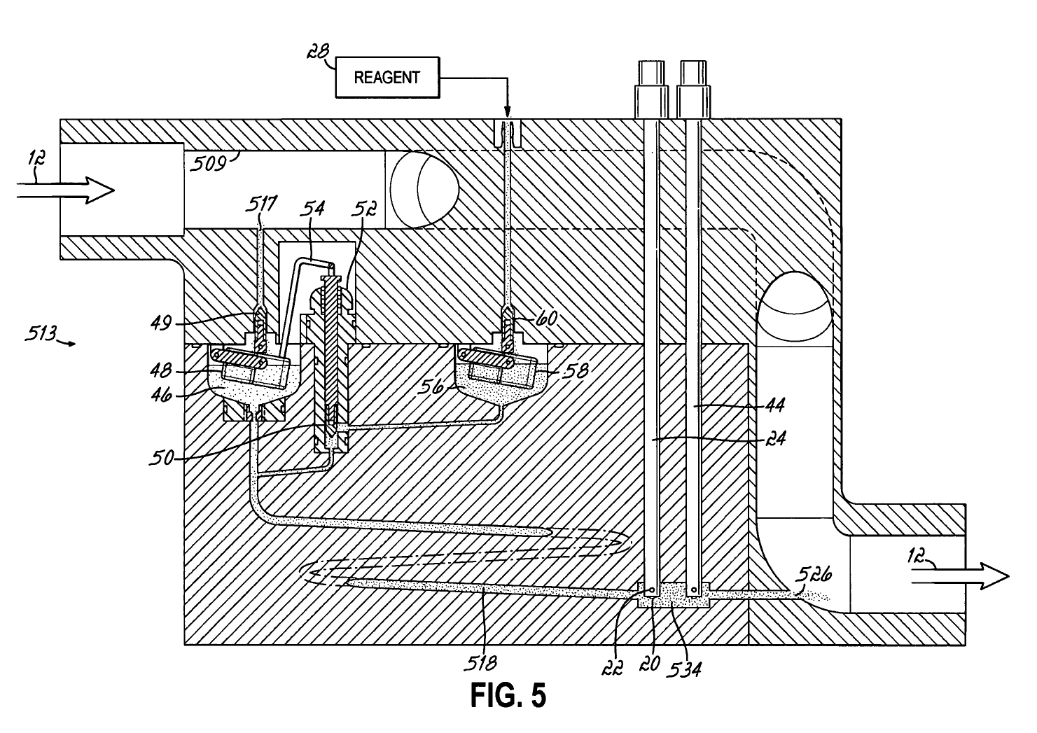

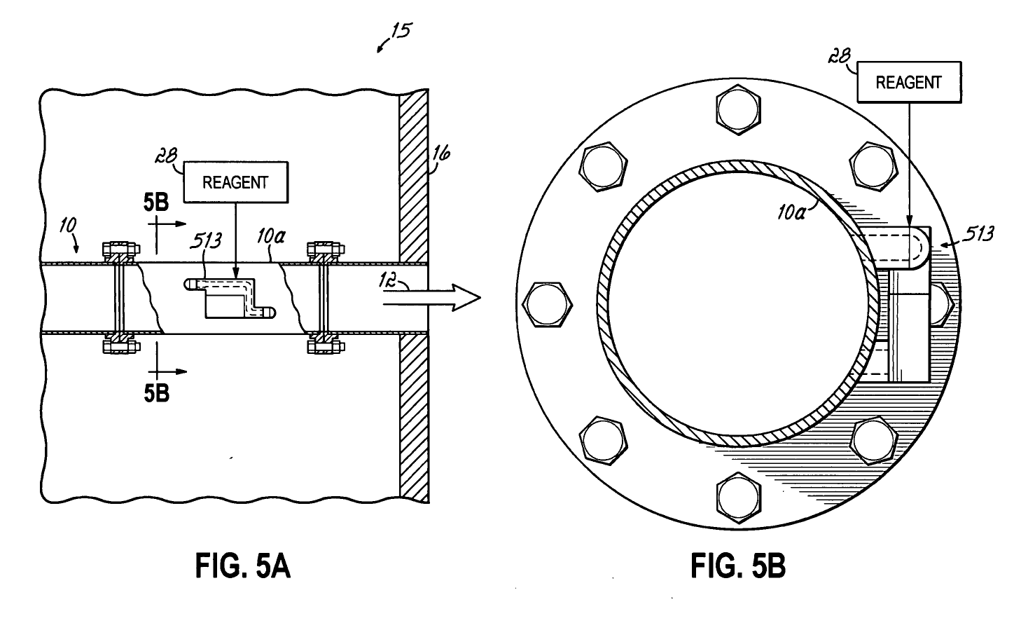

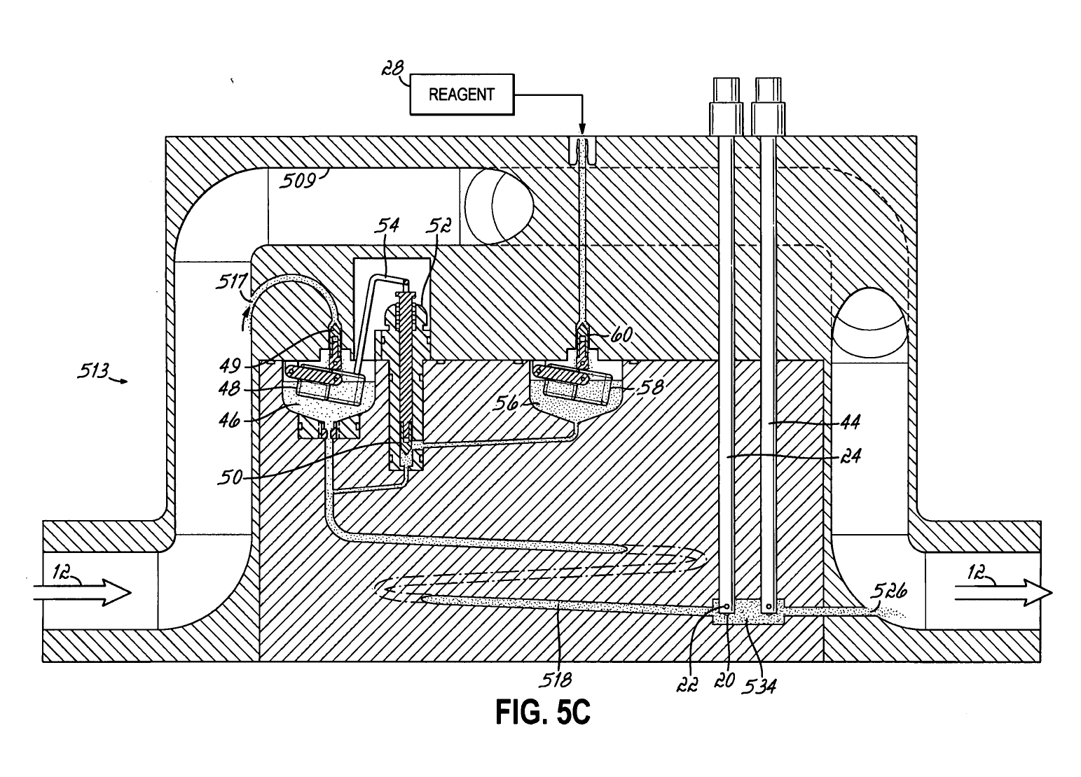

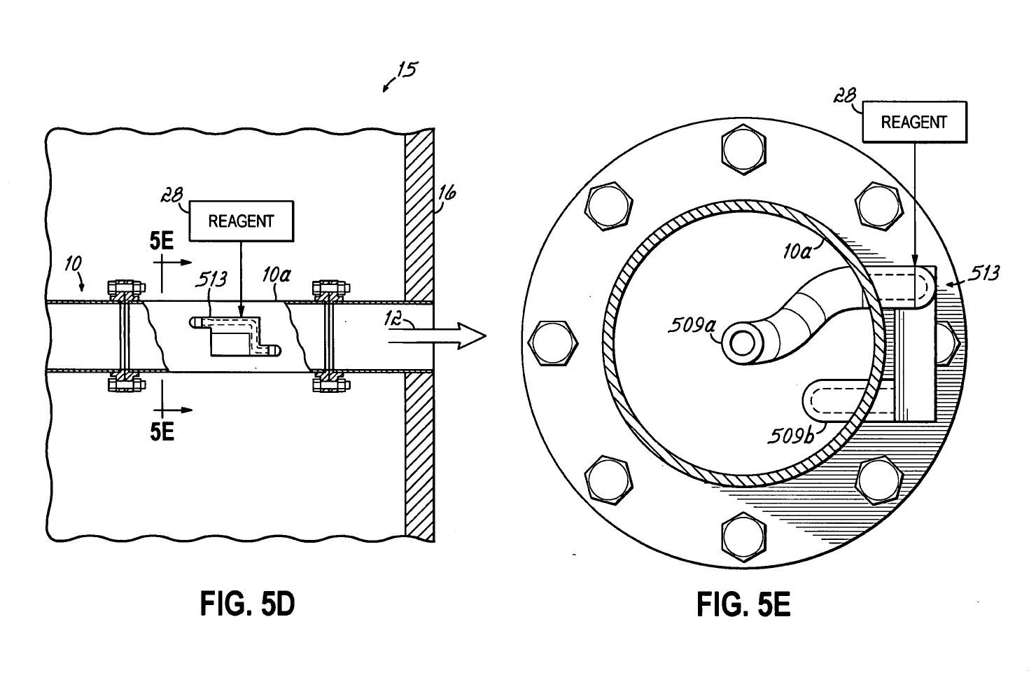

FIG. 5 illustrates an analyzer system 513 according to a fourth embodiment of the present invention, shown mounted in the duct section 10a as best seen in FIG. 5A. As the flow of the process water approaches sample inlet 517 through water flow passageway 509, a small volume of process water, i.e. the sample, enters sample inlet 517 and is directed into sample reservoir 46 containing float 48 that responds to the sample level. FIG. 5C provides an alternative configuration of water flow passageway 509 and sample inlet 517, in which the sample inlet includes a curvature in the sample passageway. This alternative arrangement may help to minimize clogging of sample inlet 517 caused by silt and other sediment settling at the mouth of the sample inlet 517, as the water flow is essentially vertically upward proximate the location of the sample inlet 517. As best shown in FIGS. 5D and 5E, this alternative configuration allows for the placement of sparge tube 509a for centerline sampling and also allows for the placement of outline 509b to be aligned with the water flow indicated by arrows 12. Float 48 is shown attached to sample reservoir 46 through a hinging mechanism. However, float 48 may also freely float within the sample reservoir 46. When the sample reservoir is full of the process water sample, the float 48 operates to close fluid communication between the sample inlet 517 and the sample reservoir 46 via a sealing valve 49.

Float 48 is operatively linked to sealing valve 50 in pneumatic valve 52, which could be an on-off valve for example, through flapper 54, and pneumatic valve 52 is fluidly coupled to reagent reservoir 56. Although reagent reservoir 56 is shown downstream of pneumatic valve 52, the relative position of reagent reservoir 56 and pneumatic valve 52 is not material to the operation of the inventive system. Reagent 28 is stored in reagent reservoir 56 from a removable liquid reagent container (not shown). As the reagent is added to reagent reservoir 56, float 58 moves to a predetermined level, at which point float 58 activates sealing valve 60 to halt the flow of reagent into reagent reservoir 56. Float 58 is shown attached to reagent reservoir 56 through a hinging mechanism. However, float 58 may also freely float within the reagent reservoir 56.

Turning back to pneumatic valve 52, when float 48 moves flapper 54, thereby activating pneumatic valve 52, reagent from reagent reservoir 56 flows into reaction zone 518 where the reagent mixes with the sample from sample reservoir 46. Reaction zone 518 conveys the mixed sample and reagent to sensor zone 534. Sensor zone 534 includes at least one sensing electrode 20 and at least one reference electrode 22, which together comprise oxidants probe 24. The flow of the mixed sample and reagent subsequently carries the mixture to outlet 526, where the mixture is expelled into the water flow passageway 509. Optionally, second probe 44 may be incorporated in the sensor zone 534. Second probe 44 may be, for example, a temperature probe or a pH probe.

FIGS. 5A and 5B show a diagrammatic view of the analyzer system 513 mounted to the outside of duct section 10a of discharge duct 10. The embodiment of the present invention shown in FIGS. 5A and 5B allows for minimal disturbance of the structural integrity of duct section 10a. In practice, installation of the analyzer system 513 as shown requires drilling two holes into duct section 10a and the welding of two stubs into those holes. The design shown in FIGS. 5A and 5B are exemplary only and are not intended to limit the scope of the present invention. One of ordinary skill in the art is capable of modifying the location of the analyzer system 513 to suit the requirements of the particular application.

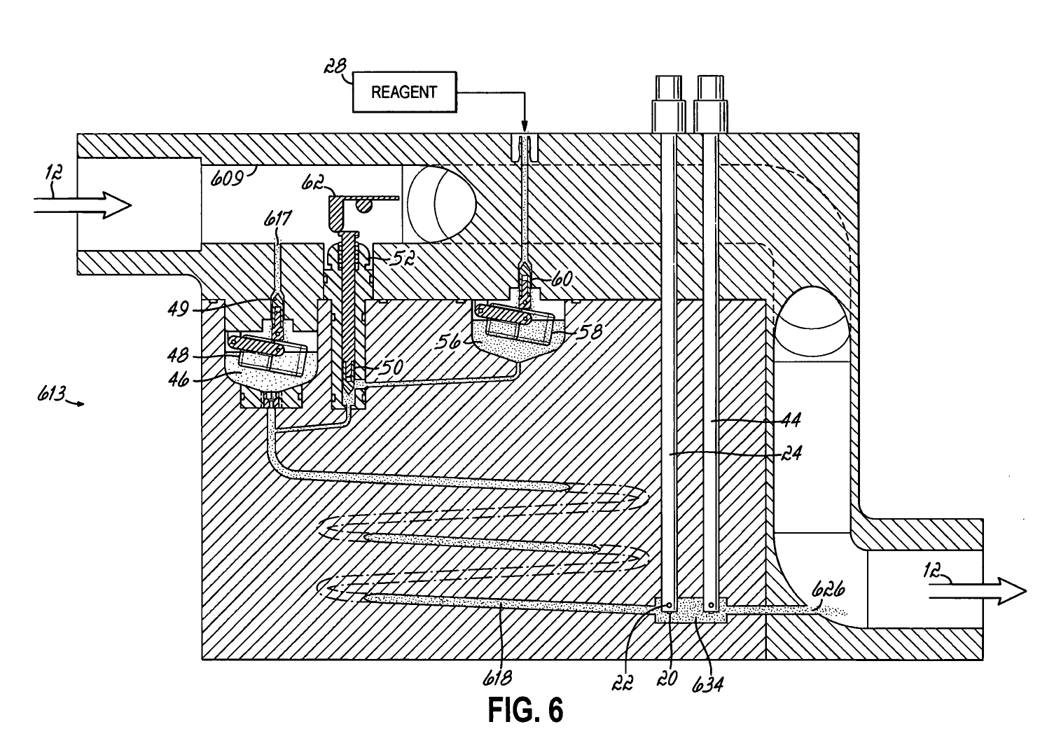

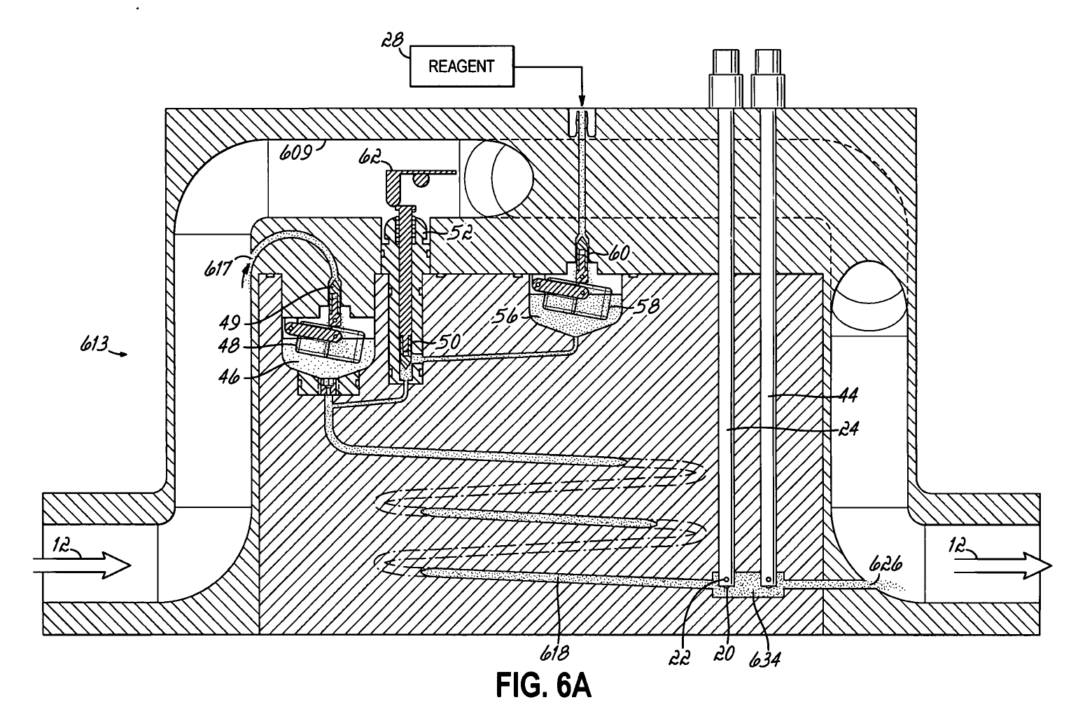

FIG. 6 illustrates an analyzer system 613 according to a fifth embodiment of the present invention, shown mounted in duct section 10a. As the flow of the process water approaches sample inlet 617, a small volume of process water, i.e. the sample, enters sample inlet 617 and is directed into sample reservoir 46. Similarly to the arrangement presented in FIG. 5C, FIG. 6A provides an alternative configuration of water flow passageway 609 and sample inlet 617, in which the sample inlet includes a curvature in the sample passageway as discussed above. The flow of sample causes actuator 62 to actuate. Actuator 62 is operatively linked to sealing valve 50 in pneumatic valve 52, and pneumatic valve 52 is in fluid communication with reagent reservoir 56. As reagent 28 is added to reagent reservoir 56, float 58 moves to a predetermined level, at which point float 58 activates sealing valve 60 to halt the flow of reagent into reagent reservoir 56. Float 58 is shown attached to reagent reservoir 56 through a hinging mechanism. However, float 58 may also freely float within the reagent reservoir 56.

Turning back to pneumatic valve 52, when actuator 62 activates pneumatic valve 52, reagent from reagent reservoir 56 flows into reaction zone 618 where the reagent mixes with the sample from sample reservoir 46. Reaction zone 618 conveys the sample and reagent to sensor zone 634. Sensor zone 634 includes at least one sensing electrode 20 and at least one reference electrode 22, which together comprise oxidants probe 24. The flow of the mixed sample and reagent subsequently carries the mixture to outlet 626, where the mixture is expelled into water duct 609. Optionally, second probe 44 may be incorporated in the sensor zone 34. Second probe 44 may be, for example, a temperature probe or a pH probe.

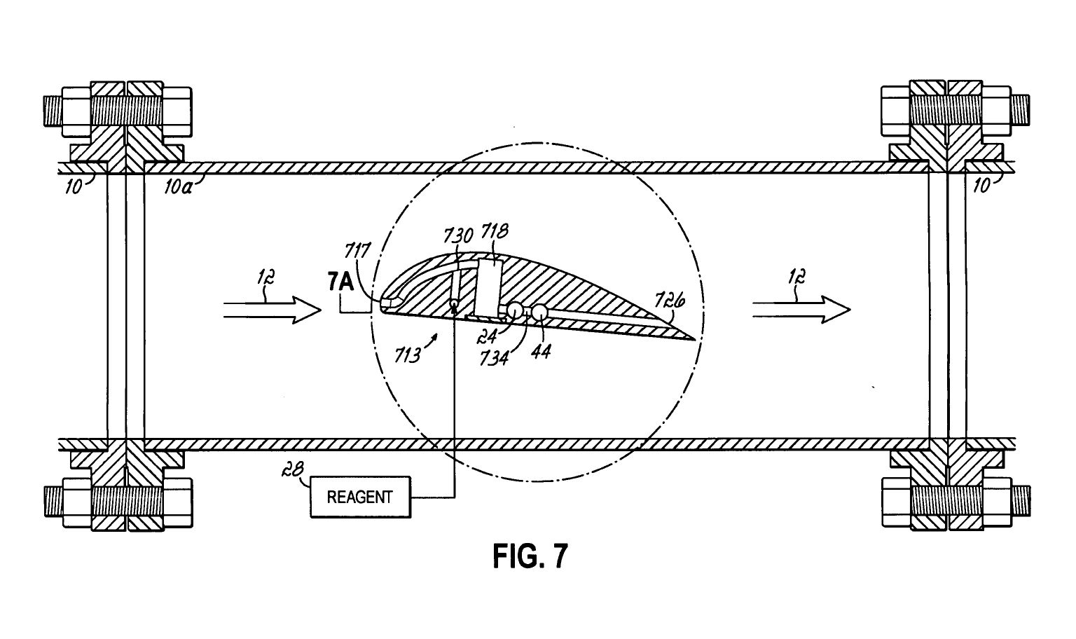

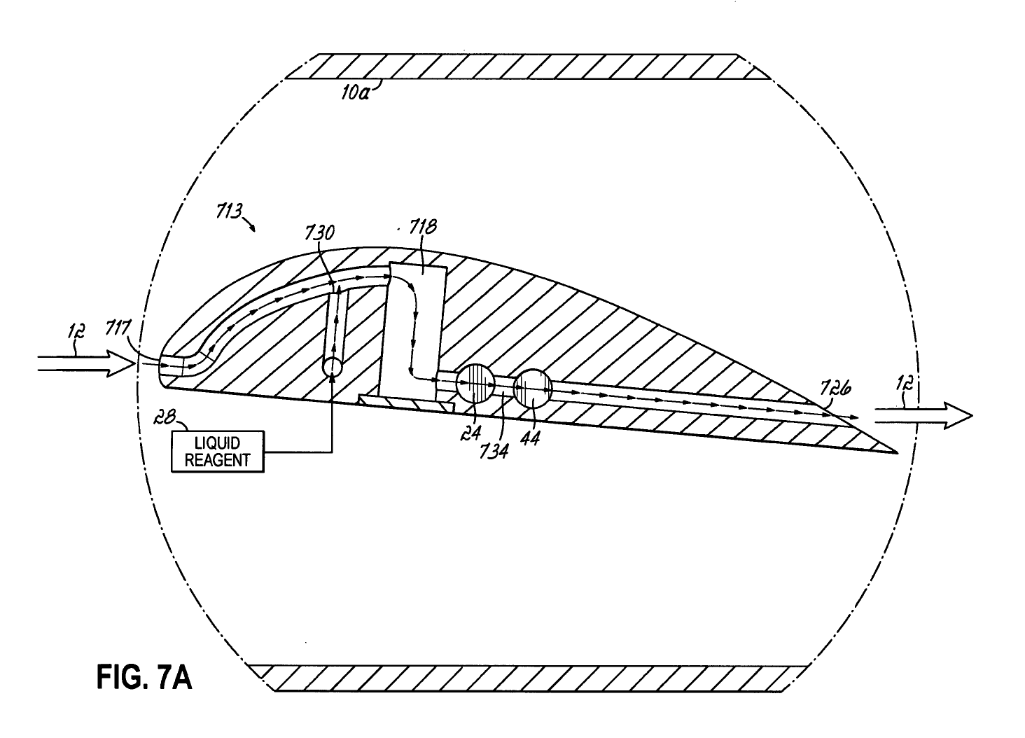

FIGS. 7 and 7A illustrate an analyzer system 713, according to a sixth embodiment of the present invention, shown mounted in duct section 10a. In one embodiment, the entire analyzer system 713 is angled downward approximately 5°, relative to a longitudinal axis of the duct section 10a, to aid in the draining of the system 713 when not in use. However, when in use, the analyzer system 713 may be horizontal and substantially parallel to the longitudinal axis of duct section 10a. Conveniently, analyzer system 713 may be mounted to duct section 10a using a pin, thereby reducing a drag on the flow of process water in duct section 10a. Mounting analyzer system 713 using a pin produces the added benefit of allowing analyzer system 713 to automatically drain when duct section 10a does not contain process water.

Returning to the operation of the embodiment shown in FIGS. 7 and 7A, as the flow of process water approaches sample inlet 717, a small volume of sample enters sample inlet 717. Concurrently, the flow of the sample and geometry of the internal fluid passageways cause reagent 28 to flow into the fluid passageway at reagent inlet 730, via dynamic pressure, to reaction zone 718. The sample and reagent mix in reaction zone 718, and the mixed sample and reagent carry the flow to sensor zone 734. Sensor zone 734 includes at least one oxidants probe 24. The flow of the sample and reagent subsequently carries the sample and reagent mixture to outlet 726, where the mixture is expelled into the duct section 10a. Optionally, second probe 44 can be incorporated in sensor zone 734. The second probe may be, for example, a temperature probe or a pH probe.

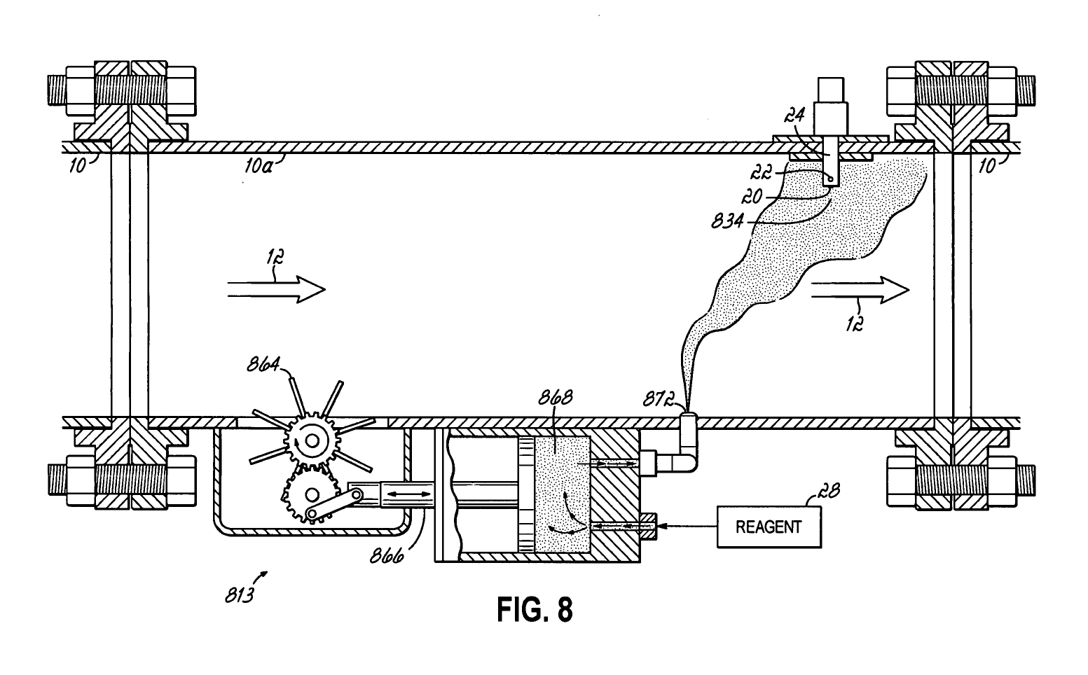

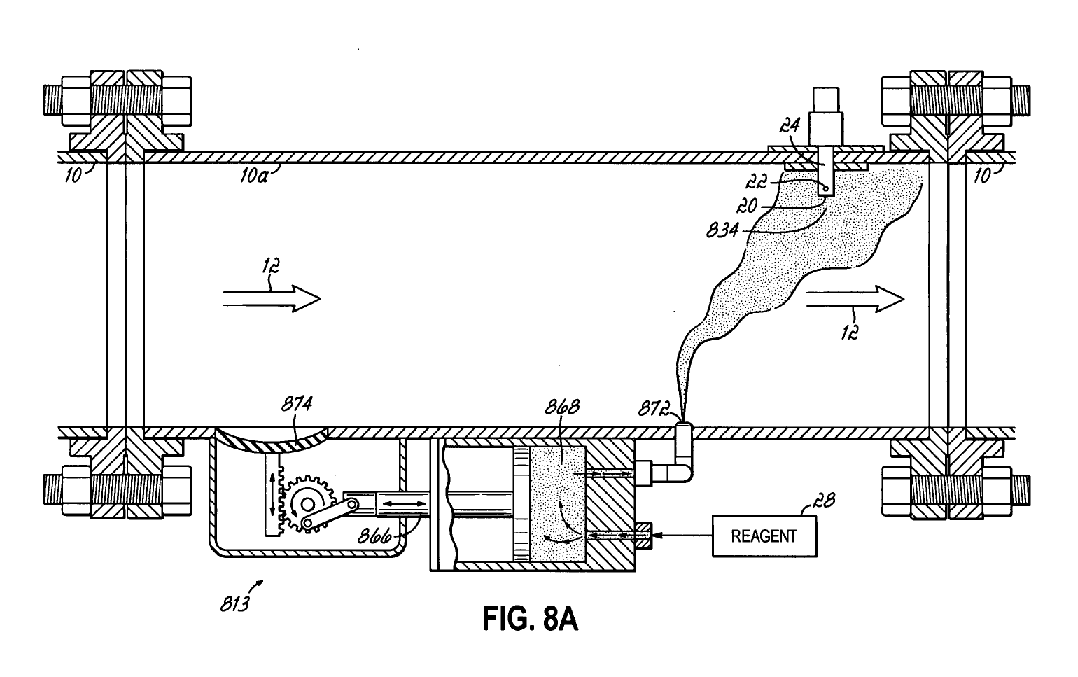

FIG. 8 illustrates an analyzer system 813 according to a seventh embodiment of the present invention, shown mounted in duct section 10a. The analyzer system in FIG. 8 operates with or without sample sipping, defined as the withdrawal a constant and known portion from a stream within a duct. For simplicity, analyzer system 813 is shown without sample sipping and the sample inlet is the entire duct section 10a. The flow of process water causes paddle wheel 864 to rotate. Paddle wheel 864 is operatively linked to piston 866 in reagent reservoir 868. Linkage is made, for instance, by a rack and pinion system. The motion of paddle wheel 864 activates reagent reservoir 868 by moving piston 866 away from outlet 872, drawing reagent 28 into reagent reservoir 868. As the flow of process water causes paddle wheel 864 to further rotate, piston 866 moves toward outlet 872, pushing reagent 28 into the duct section 10a through outlet 872.

Sensor zone 834, which is downstream of outlet 872, includes at least one sensing electrode 20 and at least one reference electrode 22, which together comprise oxidants probe 24. When reagent is added, the sample and reagent mix in the area of the duct section 10a between the outlet 872 and oxidants probe 24. In principle, the size of reagent reservoir 868 will establish an upper limit as to the amount of reagent that can be delivered to oxidants probe 24.

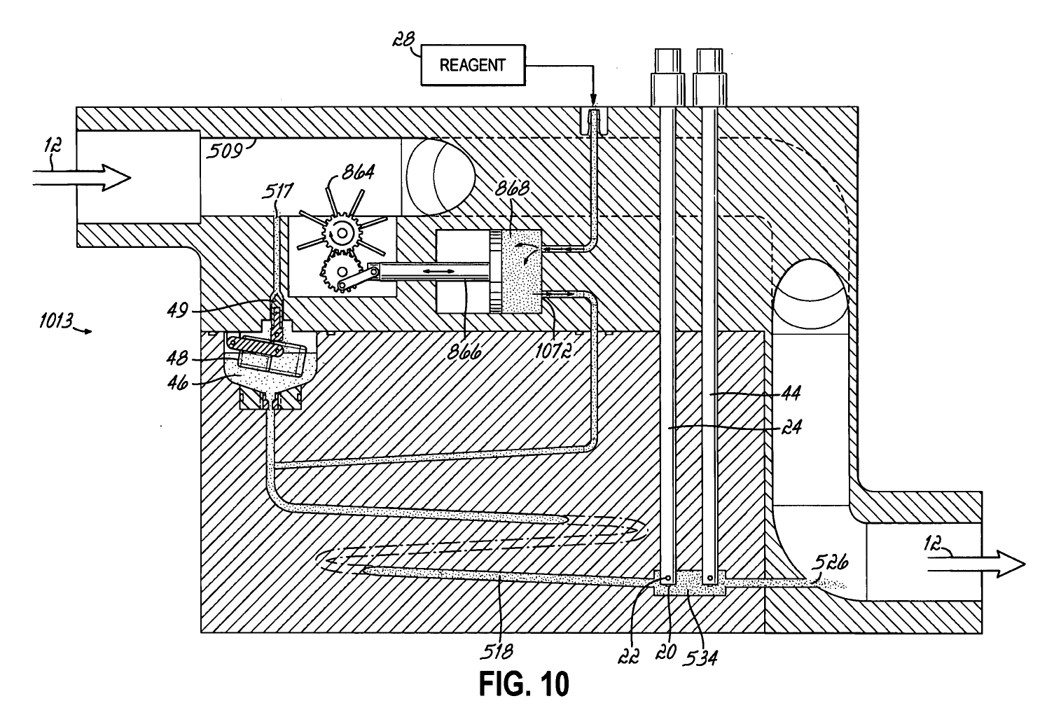

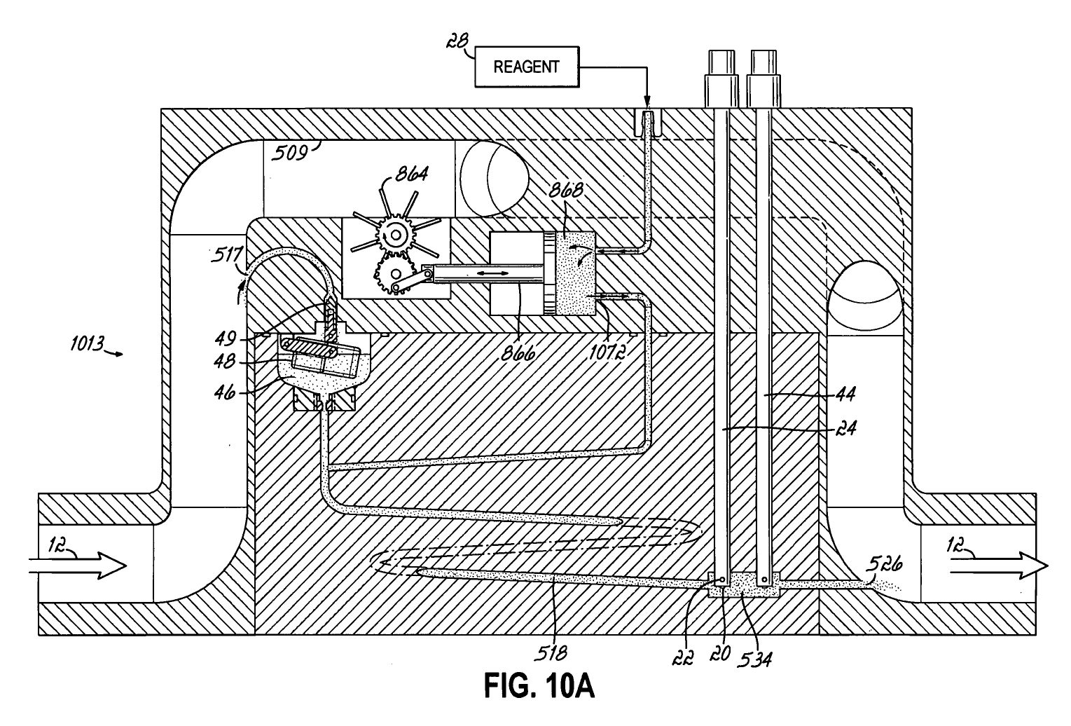

In principle, analyzer system 813 could be modified to operate with sample sipping. In such an embodiment, the sipped sample could be conveyed into duct section 10a at a point downstream of paddle wheel 864, or sample sipping could be performed upstream of paddle wheel 864, with the flow of the process water driving activity of the analyzer system. For instance, analyzer system 813 could replace actuator 62, pneumatic valve 52, sealing valve 50, and reagent reservoir 56, of FIG. 6. See FIG. 10 and related description. Once the reagent 28 is pushed into contact with the sample, both could be conveyed to reaction zone 618 to complete the sensing as discussed previously. In a further alternative embodiment of analyzer system 813, instead of duct section 10a, analyzer system 813 could be installed on a sample sipping apparatus. Stated differently, instead of the flow of process water driving the sensing of the chemical characteristic, the flow of sample itself could drive such sensing. These modifications are within the capabilities of one of ordinary skill in the art.

FIG. 8A is an alternative embodiment of the analyzer system 813 shown in FIG. 8. In FIG. 8A, paddle wheel 864 is replaced by diaphragm 874. The flow of process water, shown with arrows 12, depresses diaphragm 874, which is operatively linked to piston 866. Just as in the embodiment shown in FIG. 8, the motion of piston 866 causes the addition of reagent 28 through a one-way valve, e.g. a bellows. Thus, reagent reservoir 868 is full of reagent 28 when diaphragm 874 is not depressed, and substantially empty of reagent 28 when diaphragm 874 is fully depressed.

The embodiment shown in FIG. 8A is well-suited for the addition of a metered aliquot of a reagent and for the sequential addition of multiple reagents used for one measurement. One of ordinary skill in the art is capable of modifying the analyzer system 813, within these general principles, to suit the requirements of the particular application.

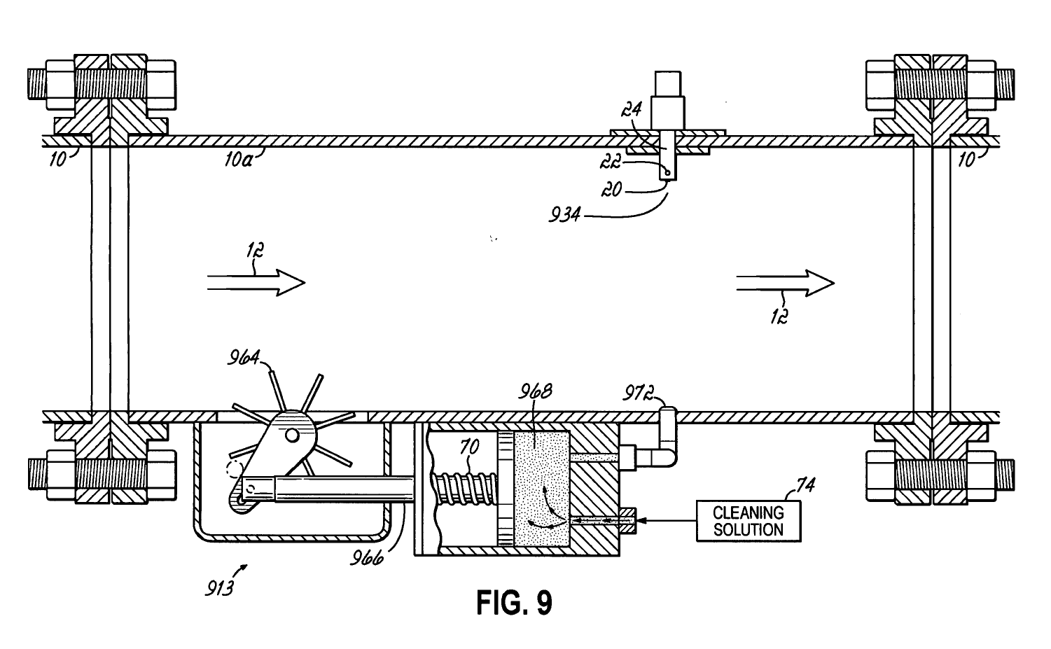

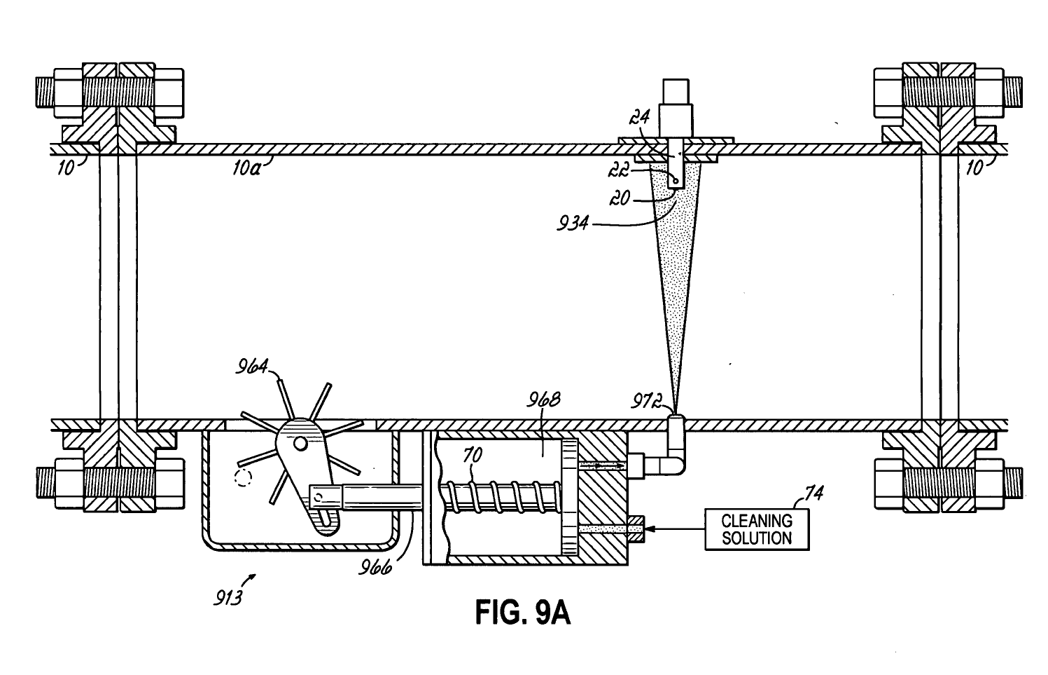

FIGS. 9 and 9A illustrate an analyzer system 913, according to an eighth embodiment of the present invention, shown mounted in duct section 10a. The analyzer system 913 in FIG. 9 operates with or without sample sipping, and is especially well-suited for providing a method for cleaning the oxidants probe 24. The flow of sample causes an actuator, such as paddle wheel 964, to rotate. Paddle wheel 964 is operatively linked to piston 966 in cleaning solution reservoir 968. Linkage is made, for instance, by a rack and pinion system. The motion of paddle wheel 964 prepares cleaning solution reservoir 968 for activation by moving piston 966 away from outlet 972. Once the flow of process water stops, piston 966 moves forward, optionally via an energy storage device such as spring 70, to blast cleaning solution 74 into the duct section 10a through outlet 972.

Sensor zone 934, which is directly above outlet 972, includes at least one sensing electrode 20 and at least one reference electrode 22, which together comprise oxidants probe 24. When cleaning solution is added, the sample and cleaning solution mix in the area of the duct section 10a between the outlet 972 and oxidants probe 24. In principle, the size of cleaning solution reservoir 968 will establish an upper limit as to the amount of cleaning solution that can be delivered to oxidants probe 24. FIG. 9A shows the delivery of the cleaning solution when the piston 966 of FIG. 9 is fully activated.

As described above, the embodiment shown in FIGS. 9 and 9A is well-suited to the delivery of cleaning solution to the sensor zone. However, the embodiment shown in FIGS. 9 and 9A can easily be added to any of the alternative embodiments described in the present application. Thus, one or more sensing measurements could be obtained while the process water flows, before or after adding cleaning solution to the sensor zone, in accordance with any of the embodiments described. One of ordinary skill in the art is capable of modifying the analyzer system 913, within these general principles, to suit the requirements of the particular application.

FIG. 10 illustrates an analyzer system 1013, according to a ninth embodiment of the present invention, shown mounted in the duct section 10a, and represents a combination of the embodiments shown in FIGS. 5 and 8. In FIG. 10, As the flow of the process water in water flow passageway 509 approaches sample inlet 517, a small volume of process water, i.e. the sample, enters sample inlet 517 and is directed into sample reservoir 46 containing float 48 that responds to the sample level. Similarly to the arrangement presented in FIG. 5C, FIG. 10A provides an alternative configuration of water flow passageway 509 and sample inlet 517, in which the sample inlet includes a curvature in the sample passageway. Float 48 is shown attached to sample reservoir 46 through a hinging mechanism. However, float 48 may also freely float within the sample reservoir 46. When the sample reservoir 46 is full of the process water sample, the float 48 operates to close fluid communication between the sample inlet 517 and the sample reservoir 46 via a sealing valve 49.

The flow of process water causes paddle wheel 864 to rotate. Paddle wheel 864 is operatively linked to piston 866 in reagent reservoir 868. Linkage is made, for instance, by a rack and pinion system. The motion of paddle wheel 864 activates reagent reservoir 868 by moving piston 866 away from outlet 1072, drawing reagent 28 into reagent reservoir 868. As the flow of process water causes paddle wheel 864 to further rotate, piston 866 moves toward outlet 1072, pushing reagent 28 through outlet 1072 into a conduit to the reaction zone 518.

Sample is released from sample reservoir 46 and reagent 28 is released from reagent reservoir 868. Both sample and reagent 28 flow into reaction zone 518. Reaction zone 518 conveys the mixed sample and reagent to sensor zone 534. Sensor zone 534 includes at least one sensing electrode 20 and at least one reference electrode 22, which together comprise oxidants probe 24. The flow of the mixed sample and reagent subsequently carries the mixture to outlet 526, where the mixture is expelled into the discharge duct 10. Optionally, second probe 44 may be incorporated in the sensor zone 534. Second probe 44 may be, for example, a temperature probe or a pH probe.

The invention harnesses the power generated by the flowing process water to drive sample collection, addition of reagent, mixing, and draining. Thus, no additional power is needed to monitor the chemical characteristic of the fluid sample, and the analyzer system can be placed at a point close to the discharge point of the process water. Because the sample is collected and analyzed close to the discharge point, minimizing sensor response time, the results obtained should be superior to any other process water analyzer systems known to the inventors.

In addition to the disclosed analyzer system, the present invention also features a method for sensing a chemical characteristic of a flowing fluid sample. The method includes, for example, providing the sample to an analyzer system; providing a reagent to the analyzer system; mixing the reagent and the sample; providing the mixed reagent and sample to a sensor zone; sensing a chemical characteristic of the sample; and removing the sample and reagent from the analyzer system. The power extracted from the flowing sample powers the steps of the method. Thus, no additional power is needed to sense the chemical characteristic of the fluid sample,

The sample is provided to the analyzer system through a sampling device capable of extracting a small sample flow rate from a large flow rate. Indeed, approximately a 1 million-fold reduction in flow rate is obtainable without additional power added to the system in the form of a pump or valve. In an embodiment of the invention, the sample inlet is a sample sipping apparatus within a process water duct. Sample sipping pertains to a design that withdraws a constant and known portion from a stream within a duct. An exemplary flow sipper in a ballast duct has an elliptical opening and is at a grazing angle relationship to the flow stream contours within the process water duct to minimize clogging and wear due to silt and other large matter, but the present invention is not intended to be so limited.

The reagent is provided to the analyzer system through any means capable of storing and delivering the appropriate amount of reagent for conducting the analysis. The term “reagent” may also include probe cleaning solution. The reagent may be a gas-phase (vapor), liquid, or solid, and its chemical composition depends upon the particular application and sensing approach used. For instance, TRO may be sensed using an iodometric approach with potassium iodide and acetic acid. Chlorine, phosphate, and silica, may be sensed using colorimetry with 2-(Diphosphonomethyl)succininc acid, vannado-molybdate, or molybdic acid, ascorbic acid, and heteropolyblue, respectively, for example. Potentiometric sensing may be used to monitor sodium, chloride, and fluoride, using diisopropylamine vapor or formic acid, for example. One of ordinary skill in the art is capable of selecting the appropriate sensing technique and associated reagent to monitor the chemical characteristic of interest, and the present invention is not intended to be limited to any particular sensing technique or reagent.

As explained above, sensing of TRO may be performed using iodometric techniques. An exemplary iodometric technique is described in U.S. Pat. No. 4,049,382, entitled “Total Residual Chlorine,” the entire contents of which are incorporated herein by reference. Briefly, the sample stream is mixed with the reagent stream containing a dissociated complex of alkali metal ion and iodide ion, along with an excess amount of iodide ion. The iodide reacts with all residual chlorine in the sample and is converted to iodine. Two probes then measure the activity of the iodine, from which the total residual chlorine is determined.

When a liquid reagent is used, for example, the analyzer system may include an actuator, such as a float and flapper, a paddle wheel, or a propeller, each of which may move in response to the flow of sample through the ballast duct. The motion of the actuator may drive a mechanism that is capable of squeezing a bladder filled with the reagent or turning a release valve. When a solid reagent is used, the solid reagent may be configured to reside in the sample stream between the sample inlet and the reaction zone.

After providing the sample and reagent to the analyzer system, the reagent and the sample are mixed. Mixing can be accomplished, for example, by flowing the reagent and sample through a reaction zone together. The reaction zone may be configured in such a way that turbulence is created while the sample and reagent are flowing through the reaction zone. For example, the reaction zone may be a coiled pipe or a fluid passageway with one or more curved portions and one or more straight portions.

The mixed reagent and sample are provided to the sensor zone at the end of the reaction zone. The sensor zone may be configured to allow automatic drainage of the mixed sample and reagent away from the sensor after the sensor has sensed the chemical characteristic of the mixed sample and reagent. For example, the reaction zone may be arranged on a negative incline, and the sensor zone may continue that negative incline. In an alternate embodiment, the reaction zone may end with a slightly upward portion into the sensor zone, and the sensor zone may then be followed by a slightly downward flowing portion. A cross section of the latter embodiment would resemble an inverted U-shape with the sensor zone at the pinnacle of the inverted U.

The analyzer system is configured in a manner that allows the outflow of the mixed sample and reagent after sensing the chemical characteristic of the fluid sample. To prevent the need for additional energy input, the geometry of the system can be configured to take advantage of gravity and flow velocity. For instance, the sample inlet, reagent inlet, reaction zone, sensor zone, and system outlet, can all be arranged on a negative incline, with each successive zone spatially lower than the preceding zone. In the alternate embodiment discussed above, the inverted U-shape would cause the mixed sample and reagent to flow away from the sensor zone. In the latter embodiment, the inverted U can terminate at or near the process water outflow, which contains the remaining process water that was not collected in the sample inlet. Alternatively, the analyzer system may be configured to take advantage of pressure dynamics to draw the mixture away from the sensor zone.

Additionally, the embodiments shown above return the mixed sample and reagent to the process water duct, which is a feasible solution to waste-stream generation when the reagent is not particularly hazardous. However, the invention is not limited to only such waste reinjection. In situations where a hazardous material, such as chromium or mercury, is used as the reagent, one of ordinary skill in the art is capable of modifying the embodiments shown to allow for collection of a waste stream to hold for proper disposal.

While the various principles of the invention have been illustrated by way of describing various exemplary embodiments, and while such embodiments have been described in considerable detail, there is no intention to restrict, or in any way limit, the scope of the appended claims to such detail. Additional advantages and modifications will readily appear to those skilled in the art.

As various changes could be made in the above-described aspects and exemplary embodiments without departing from the scope of the invention, it is intended that all matter contained in the above description shall be interpreted as illustrative and not in a limiting sense.

What is claimed is:

1. An analyzer system for sensing a chemical characteristic of a fluid sample comprising:
a sample inlet configured to receive the sample via the sample inlet;
a reagent inlet configured to receive a reagent via the reagent inlet;
a reaction zone in fluid communication with the sample inlet and the reagent inlet and being configured to receive the sample from the sample inlet and the reagent from the reagent inlet for mixing the sample with the reagent; and
a sensor zone in fluid communication with the reaction zone,
wherein the sensor zone comprises a sensor configured to sense the chemical characteristic of the mixed sample and reagent, and
further wherein the sensor zone is configured to allow automatic drainage of the mixed sample and reagent away from the sensor after the sensor has sensed the chemical characteristic of the mixed sample and reagent.

2. The analyzer system of claim 1, further comprising a sample reservoir in fluid communication with the sample inlet for receiving the sample via the sample inlet and a reagent reservoir in fluid communication with the reagent inlet for receiving the reagent via the reagent inlet.

3. The analyzer system of claim 1, wherein the sensed chemical characteristic comprises the total residual oxidants present in the mixed sample and reagent.

4. The analyzer system of claim 1, wherein the reagent is a solid reagent or a liquid reagent.

5. The analyzer system of claim 1, wherein the reagent is a gas-phase reagent.

6. An analyzer system for sensing a chemical characteristic of a fluid sample comprising:
a sample inlet configured to receive the sample via the sample inlet;
a reagent inlet configured to receive a reagent via the reagent inlet;
a sample reservoir in fluid communication with the sample inlet for receiving the sample from the sample inlet;
an actuator having a first position and a second position, the actuator being configured to move from the first position to the second position in response to the sample being received in the sample reservoir;
a reagent reservoir in fluid communication with the reagent inlet for receiving the reagent from the reagent inlet;
a valve operatively coupled to the actuator being configured to release the reagent from the reagent reservoir;
a reaction zone in fluid communication with the sample reservoir and the reagent reservoir and being configured to receive the sample from the sample reservoir and the reagent from the reagent reservoir for mixing the sample with the reagent; and
a sensor zone in fluid communication with the reaction zone,
wherein the sensor zone comprises a sensor configured to sense the chemical characteristic of the mixed sample and reagent, and
further wherein the reagent regulator is configured to release the reagent from the reagent reservoir in response to movement of the actuator from the first position to the second position.

7. The analyzer system of claim 6, wherein the reagent inlet is located downstream of the sample inlet.

8. The analyzer system of claim 6, wherein the reagent reservoir is located downstream of the sample reservoir.

9. The analyzer system of claim 6, wherein the reaction zone is located downstream of both the sample reservoir and the reagent reservoir.

10. The analyzer system of claim 6, wherein the sensor zone is located downstream of both the sample reservoir and the reagent reservoir.

11. The analyzer system of claim 6, wherein the actuator comprises a float.

12. The analyzer system of claim 6, wherein the sensed chemical characteristic comprises the total residual oxidants present in the mixed sample and reagent.

13. An analyzer system for sensing a chemical characteristic of a fluid sample comprising:
a sample inlet configured to receive the sample via the sample inlet;
a reagent inlet configured to receive a reagent via the reagent inlet;
an actuator having a first position and a second position, the actuator being configured to move from the first position to the second position in response to sample flow;
a reagent reservoir in fluid communication with the reagent inlet for receiving the reagent from the reagent inlet;
a valve operatively coupled to the actuator;
a reagent outlet in fluid communication with the reagent chamber;
a reaction zone in fluid communication with the sample inlet and the reagent outlet and being configured to receive the sample from the sample inlet and the reagent from the reagent outlet for mixing the sample with the reagent; and
a sensor zone in fluid communication with the reaction zone,
wherein the sensor zone comprises a sensor configured to sense the chemical characteristic of the mixed sample and reagent, and
further wherein the valve is configured to release the reagent from the reagent chamber via the reagent outlet in response to movement of the actuator from the first position to the second position.

14. The analyzer system of claim 13, wherein the sensed chemical characteristic comprises the total residual oxidants present in the mixed sample and reagent.

15. The analyzer system of claim 13, wherein the actuator comprises a paddle wheel, a diaphragm, or a propeller.

16. The analyzer system of claim 13, wherein the valve comprises a piston and the reagent reservoir comprises a piston chamber.

17. The analyzer system of claim 13, wherein the valve comprises a transmission.

18. The analyzer system of claim 13, wherein the reagent outlet comprises a spray nozzle.

19. The analyzer system of claim 13, wherein the analyzer system is a component of a ballast water pipe and wherein the reaction zone is a region of said ballast water pipe downstream of the reagent outlet and upstream of the sensor zone.

20. The analyzer system of claim 13, wherein the sample inlet is an opening in the ballast water pipe in a region upstream of the actuator and wherein the actuator is upstream of the reagent outlet.

21. A method for monitoring total residual oxidants present in a flowing fluid sample comprising:
providing the sample to the analyzer system of claim 1 or claim 6 or claim 13 via the sample inlet;
providing the reagent via the reagent inlet;
mixing the sample and the reagent in the reaction zone;
providing the mixed sample and reagent to the sensor zone; and
sensing the total residual oxidants present in the sample.

22. The method of claim 21, wherein power generated from the flowing fluid powers the method.
